(19) United States
(12) Patent Application Publication
(10) Pub. No.: US 2011/0275077 A1
(43) Pub. Date: Nov. 10, 2011

(54) Oligonucleotide-Coated Affinity Membranes and Uses Thereof

(76) Inventors: William James, Potomac, MD (US); Vladimir Rait, Rockville, MD (US)

(21) Appl. No.: 13/143,935

(22) PCT Filed: Jan. 11, 2010
(86) PCT No.: PCT/US10/20680

§ 371 Date: Jul. 11, 2011

Related U.S. Application Data
(60) Provisional application No. 61/193,946, filed on Jan. 12, 2009.

(51) Int. Cl. C12Q 1/68; C08G 73/10; C08G 64/42; C07H 21/00; C08G 63/91

(52) U.S. Cl. 435/6.11; 536/22.1; 536/25.4; 525/54.2

(57) ABSTRACT

A method of analyzing tissue sections in a manner that provides information about the presence and expression levels of multiple biomarkers at each location within the tissue section. The method comprises the preparation of membranes having covalently bound oligonucleotides and the use of those membranes for evaluation of various markers in the sample. The membranes may be arranged in stacks, wherein each layer has a different oligonucleotide capture strand. Transfer oligonucleotides complementary to the capture strands are attached through a cleavable bond to antibodies that recognize and bind to specific biomarkers present in the tissue sample. The tissue sample is exposed to the antibody-transfer strand conjugate and then treated with a cleaving reagent. Upon cleavage, the transfer strand migrates through the stack and binds to the capture strand. The level of expression of the biomarker may be determined by measuring expression of a reporter on the transfer strand.

### CROSS-REFERENCE TO RELATED APPLICATION

This application is a continuation in part, and claims the benefit of, U.S. Patent Application No. 61/193,946 filed Jan. 12, 2009. That application is incorporated herein in its entirety.

### GOVERNMENT SUPPORT

This invention was made with government support under Cooperative Agreement #70NANB7H7028 awarded by the U.S. Department of Commerce, National Institute of Standards and Technology. The U.S. Government has certain rights in the invention.

### BACKGROUND

Membranes are employed in a wide variety of biological assays and in-vitro diagnostics products in laboratory, field, and point-of-care settings. These include, for example, various gel blotting procedures as well as “dip-stick” lateral flow tests for pathogens and other analytes. These devices and approaches typically employ fibrous membranes made from nitrocellulose or PVDF.

More recently, an alternative to fibrous membranes has been employed for a variety of diagnostics and biodetection applications. These membranes, known as “track-etched” membranes (“TEMs”) comprise thin films with discrete pores that are formed through a combination of charged-particle bombardment (or irradiation) followed by chemical etching (see photograph FIG. 17). The particle bombardment results in the formation of damaged areas in the film (tracks) which are subsequently etched to form pores with a defined size. Recent uses of TEMs in various biological assays are described for example by Hanot et al. in “Industrial applications of ion track technology,” Nucl. Instrum. Methods Phys. Res. Sect. B, 267: 1019-1022 (2009) and by Jones et al. in “Expanding the use of track-etched membranes,” IVD Technology November/December/2002. The authors describe the employment of TEMs in biosensors, cytology, bacterial detection, and a variety of other biological fields. The aforementioned references describe use of “off the shelf” TEMs in their native form. A few other applications use TEMs as substrates that are coated with various probes and capture moieties. For example U.S. Pat. No. 5,968,745 to Thorp et al. describes a polymer electrode for detecting nucleic acid hybridization that that utilizes oligonucleotide probes bound to a single track-etched membrane via carbodiimide condensation.

One highly innovative use of TEMs for the analysis of tissue sections is the “Layered Peptide Array” (“LPA”) as described by Emmert-Buck, et al. in U.S. Patent Application Pub. No. 2009/0215073 A1 (application Ser. No. 12/289,736) and in Clinica Chimica Acta 376 (2007) 9-16 and the Journal of Molecular Diagnostics, Vol. 9, No. 3, July 2007 pgs. 297-304 (each of these references are incorporated herein in their entirely). An LPA is a stack of TEMs, each of which is coated with a different peptide or protein antigen; each layer in the stack has a specific affinity to a different antibody. Antibodies to target proteins are applied to the tissue section in much the same manner as in immunohistochemistry. After washing, the antibodies are released from the tissue section and passed vertically through the peptide-coated TEMs while maintaining their two-dimensional position. The antibodies are specifically captured by the target layer to which a mimic of the natural target antigen has been coated. Alternatively, Emmert-Buck et al. disclose use of a cocktail of conjugated antibodies (a primary antibody attached to a transfer or “shuttle” antibody) that can be applied to the tissue; the shuttle antibody is then cleaved and captured on a complementary affinity ligand coated upon a layer of the stack. The layers of the stack are then separated, and the transfers are read.

The Layered Peptide Array approach, which is a subset of related techniques known in the literature as “Layered Expression Scanning (LES)”, significantly increases the number of markers quantifiable per tissue section. (See U.S. Pat. Nos. 7,214,477, 6,969,615, 6,602,661, U.S. patent application Ser. No. 11/189,038; Englert C R et al., Layered expression scanning: rapid molecular profiling of tumor samples. Cancer Res. 2000; 1526-30; Tangrea M A et al., Layered expression scanning: multiplex analysis of RNA and protein gels. Biotechniques 2003; 1280-5; Gillespie J W et al. Molecular profiling of cancer. Toxicol. Pathol. 2004; 67-71; Galperin M M, et al., Multimembrane dot-blotting: a cost-effective tool for proteome analysis. Biotechniques 2004; 1046-51; Gannot G et al. Layered peptide arrays: high-throughput antibody screening of clinical samples. J. Mol. Diagn. 2005; 427-36; Patel V et al. Profiling EGFR activity in head and neck squamous cell carcinoma by using a novel layered membrane Western blot technology. Oral Oncol. 2005; 503-8; all incorporated herein by reference).

Importantly, LPAs and LES permit the analysis of multiple biomarkers in various 2-D samples such as tissue sections while preserving the localization of these biomarkers. In other words, when used with tissue sections this approach combines classical pathology with multiplex array based technologies. These techniques address important unmet needs in the emerging field of “Personalized Medicine”—the development and use of therapies specifically targeted to the disease characteristics of individual patients is widely predicted to be the key driver of 21^{st} century medicine. Better diagnostics linked to drugs are anticipated to significantly improve patient outcomes while reducing healthcare costs by avoiding prescriptions of expensive drugs that prove ineffective for many patients. Many of the new targeted therapies that have come to market in recent years cost over $75,000 per patient per year but are effective in only a narrow subset of patients (˜15%) to whom they are administered. Thus, there is an urgent and compelling need for new diagnostic tests that can accurately predict whether a particular targeted therapy will work for a particular patient. Unfortunately, neither conventional techniques for tumor analysis nor newer detection technologies have proven adequate to meet this need. These techniques typically fail either in their multiplex capability or their ability to preserve the shape and morphology of the tissue section which is often needed for accurate diagnosis.

Newer multiplex technologies such as DNA microarrays or mass spectrometry (MS), as well as older ELISA based techniques, require that samples be homogenized. Yet, a typical biopsy sample would present only a small minority of infiltrating tumor cells of interest. Those cells would be surrounded by numerous other types of cells (normal cells, fibroblasts, lymphocytes, vascular cells etc.) presenting dissimilar gene expression and cell signaling profiles, which are potential biomarkers and drug targets. In a visual field of an invasive tumor, an important issue to a pathologist is whether the tumor cell population is molecularly homogeneous or whether there exist sub-clones within it with distinct transcriptomic or proteomic profiles (Uhlen M et al., 2005, A human protein atlas for normal and cancer tissues based on antibody proteomics. Mol. Cell Proteomics. 4:1920-1932, incorporated herein by reference).

Very small tissue samples such as core needle biopsies are currently being taken from small tumors for performing a tumor-specific molecular diagnosis to enable personalized drug treatments. Additionally, advancement of personalized medicine has been stymied by a paucity of technologies that can effectively derive predictive biomarkers from diseased tissues. In the case of cancer, there is a particular need for techniques that can profile multiple biomarkers in tumor sections, since most of the newer targeted therapies interact with numerous signaling proteins (Faivre S et al., New paradigms in anticancer therapy: targeting multiple signaling pathways with kinase inhibitors. Semin. Oncol. 2006; 407-20, incorporated herein by reference.).

It would therefore be desirable to have an approach for analyzing multiple biomarkers in tissue and other biological samples that overcomes the aforementioned limitations of the LPA method by providing TEMs coated with capture probes that remain more permanently bound thereto, that can be manufactured in an efficient, cost-effective, and reproducible manner, and that can be engineered for the properties of highly specific ligand binding in predictable and variable ways.

### SUMMARY

Disclosed is method of analyzing tissue sections (and other 2-D samples) in a manner that provides information about the presence and expression levels of multiple biomarkers (or other targets) at each location within the tissue section.

In short, the method utilizes a plurality or stack of permeable layers (TEMs or gels) each having a specific oligonucleotide (capture strand) covalently bound thereto so as to create affinity layers. A plurality of antibodies (primary or secondary) are also provided, each of which is conjugated via a cleavable bond to an oligonucleotide (transfer strand) that is complementary to the capture strand. A fluorophore or other detectable moiety may be attached to the transfer strand.

These conjugated antibodies are applied to the tissue section (or other sample of interest) and unbound antibodies are washed or otherwise removed. The affinity membrane stack is then applied to the tissue section. The transfer oligonucleotide is then cleaved from the conjugate antibody and migrates through the stack until the transfer oligonucleotide hybridizes to the layer coated with its complementary capture strand.

When used with tissue sections, the layers may then be analyzed by a number of imaging modalities to generate data showing the presence and expression levels of multiple biomarkers (or other targets) at a given location within the tissue section.

Included in the disclosure is a method of covalently binding fixed oligonucleotides to TEM layers that prevents their migration to adjacent layers when the transfer of corresponding transfer oligonucleotides through the stack is underway.

Also disclosed are methods that use capture oligonucleotides conjugated to layers that are formed from transparent, hydrophilic, polymeric hydrogel types of materials, such as those of natural origin (e.g. an agarose) or of synthetic origin (e.g. a polyacrylamide).

Also disclosed are methods of imaging the processed layers following transfer including a method for the serial analysis of an entire stack that need not be separated.

Also disclosed are methods of analyzing images generated with the present invention using software and the like in a manner that, when used with tissue sections, can assist a clinician with medical decision making.

With the foregoing and other objects, advantages, and features of the disclosure that will become hereinafter apparent, the nature of the disclosure may be more clearly understood by reference to the following detailed description of the disclosure, the appended claims and to the several views illustrated in the drawings.

### BRIEF DESCRIPTION OF THE DRAWINGS

Other objects and advantages of the present disclosure will be evident from the following detailed description, with like reference numbers referring to like items throughout.

FIG. 1 provides enlarged perspective views of the affinity membranes (frame A) as well an enlarged illustration of the antibody-oligonucleotide conjugates (frame B) and use of the same for analysis of tissue sections (frames C-E).

FIG. 2 is an enlarged illustration showing typical antibody-oligonucleotide conjugates that may be used with the affinity membranes disclosed herein.

FIG. 3 shows the chemical structures of three polymers commonly used as substrates for making commercially available track-etched membranes.

FIG. 4 is an illustration showing conversion products after etching of a polyester [poly(ethylene terephthalate)], a polycarbonate, or a polyimide to an oxoacid (B) in the course of the alkaline etching. Two kinds of functional groups are formed from the polymer: carboxylic acid end groups and alcohol end groups.

FIG. 5 (A) shows the reaction of an aminated oligodeoxynucleotide which may be primary or secondary with an oxoacid of a track-etched poly(ethylene terephthalate) via carbodiimide condensation. (B) shows an example of that reaction with a polycarbonate membrane.

FIG. 6 illustrates the structure of one of oligodeoxynucleotides used in EXAMPLES. The 24-mer (named ATP17) bears a primary amino group, attached through a particular spacer to the 3′ end, and a fluorescent label, Cy5, attached to the oligodeoxynucleotide's 5′ end. Other used modifications are also shown.

FIG. 7 is a bar graph with the results of a reaction in which a transfer oligonucleotide was used to probe for a hybridized interaction with a capture oligonucleotide coated on track-etched poly(ethylene terephthalate) membranes in the presence (A) or absence (B) of a water-soluble carbodiimide, EDC.

FIG. 8 is a bar graph showing the results of oligonucleotide (ATP17) interaction with track-etched polycarbonate membranes in the presence (A) or absence (B) of a water-soluble carbodiimide, EDC.

FIG. 9 is a bar graph comparing the amounts of capture oligonucleotides that can be adsorbed or covalently coupled to Polyimide, poly(ethylene terephthalate) and polycarbonate track etched membranes.

FIG. 10 is a bar graph showing the results of the joint (A) and sequential (B) incubation of the track-etched polycarbonate membranes with ATP17 and EDC. Bars correspond to the red fluorescence intensity detected either in the complete incubation mixture (A1) or in a membrane withdrawn after the incubation from the mixture contained only ATP17 (B1), in membrane sequential washings with MES buffer (2-4), 10% acetonitrile (5-7), 6×SSPE buffer (8), and in a pair of the resulting membranes (9 and 10).

FIG. 11 shows fluorescent images of track-etched polycarbonate membranes coated with ATP17 according to the joint (A) or sequential (B) procedure of the oligodeoxynucleotide immobilization. Graphs C and D characterize the fluorescence intensity distribution along diameters of membranes A and B respectively.

FIG. 12 is a bar graph comparing ATP25 interactions with the plain track-etched polycarbonate membrane (A) and the membrane with the immobilized complementary ATP60 (B). Bars correspond to the red fluorescence intensity detected in the membranes after washings with 6×SSPE buffer (1), in the last washing with that buffer (2), in following washings with 10% (3-5) and 30% (6) acetonitrile, and in the resulting membranes (7) washed again with the SSPE buffer.

FIG. 13 is a bar graph showing results of the thermal dissociation of a complementary duplex formed by the fluorescein-labeled ATP95 and ATP17 immobilized on the track-etched polycarbonate membrane. Bars correspond to the green fluorescence intensity detected in the membrane upon hybridization and washings with 6×SSPE buffer (1), released at 60° C. into 25 mM phosphate buffer, pH 7 (2) and following hot washings with the buffer (3 and 4), and retained on the membrane (5).

FIG. 14 illustrates the results of an induced release of a the Cy5-labeled oligodeoxynucleotide induced from an antigen-antibody-streptavidin construct and the oligonucleotide spontaneous distribution in a stack of membranes that contained single membrane with the complementary immobilized oligonucleotide and two membranes with non-complementary oligonucleotides. Fluorescent images of membranes (0-8) and Whatman 3MM paper (9) kept in a stack for 1 hr at 45° C. are shown. Numbers below images correspond to positions of the membranes in the stack. 0—Track-etched, coated with poly(vinylpyrrolidone) polycarbonate membrane; 1—irregular shaped track-etched polycarbonate membrane with a complex composed of the adsorbed rabbit IgG, goat anti-rabbit Ab conjugated with streptavidin, and transfer oligonucleotide ATP73; layers 2, 4, 6, and 8—as 0; 3, 5, and 7—track-etched polycarbonate membranes with the immobilized capture oligonucleotides ATP62, ATP61, and ATP 60, respectively. The Whatman filter was wetted with 4×SSC buffer contained 0.1% SDS, and 25 mM TCEP.

FIG. 15 is an illustration showing serially imaging of a transparent membrane stack that may be optionally used with the affinity membranes disclosed herein.

FIG. 16 is a scanning electron micrograph of a typical track-etched membrane.

FIG. 17 is a set of image data and quantitative analysis to demonstrate a comparison of non-multiplex to 3-fold multiplex analysis of three target analytes in a pathology tissue section, and includes a quantitative analysis of the intensity of signals in the images.

FIG. 18 is a set of image data and quantitative analysis to demonstrate a comparison of 2-fold multiplex to 3-fold multiplex analysis of three target analytes in a pathology tissue section, and includes a quantitative analysis of the intensity of signals in the images.

FIG. 19 is a demonstration of the replication of an intensity pattern of an assay target in a tissue specimen on three detection layers after transfer of transfer oligonucleotides, and includes a quantitative analysis of the intensity of signals in the images.

### DETAILED DESCRIPTION OF A PREFERRED EMBODIMENT

1. Overview

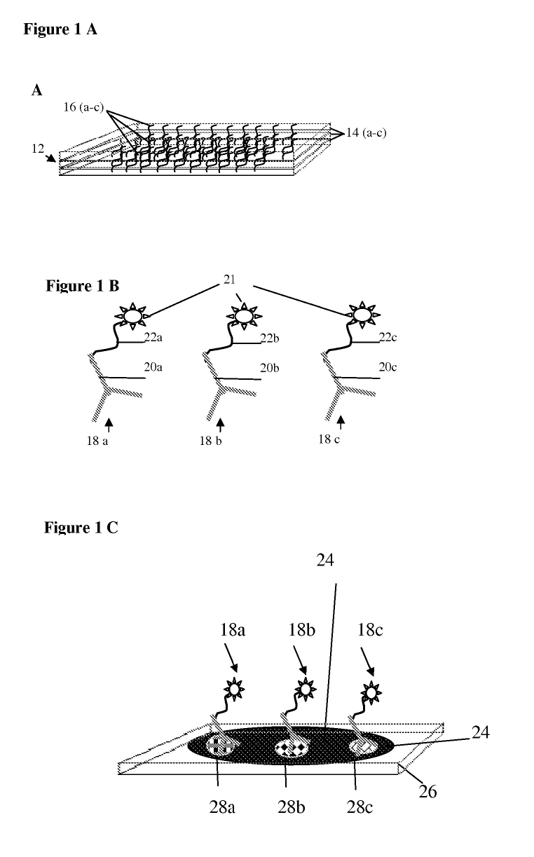

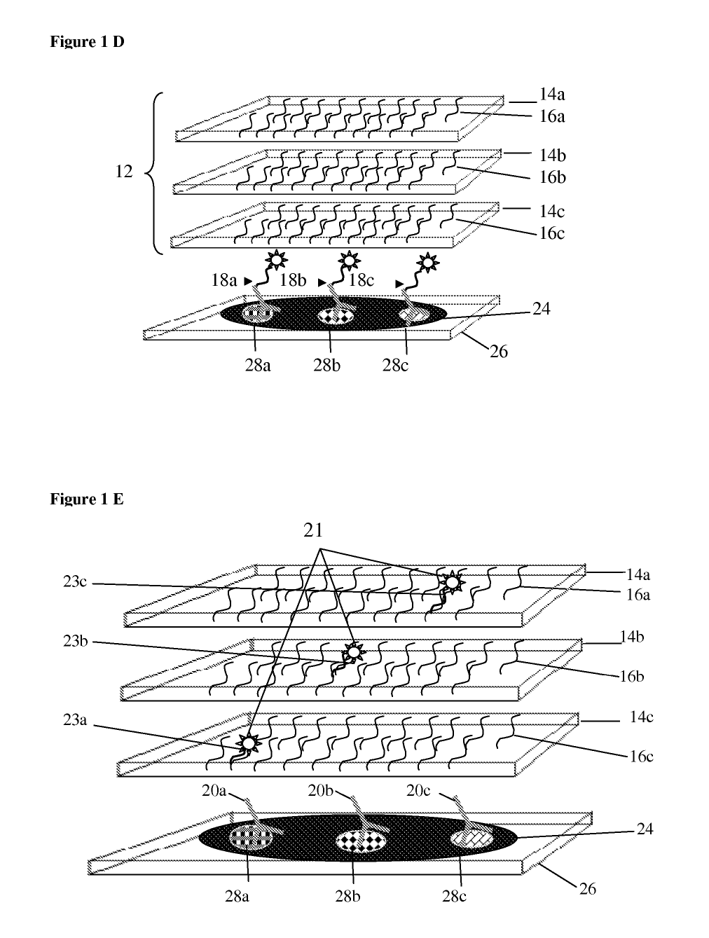

A preferred embodiment of the present invention is illustrated in FIG. 1. With reference to frames A and B of FIG. 1 this embodiment preferably includes an affinity membrane stack 12 which comprises multiple layers of track-etched membranes (“TEMs”) 14 (a-c) each coated with a different, specific oligonucleotide 16 (a-c), typically a capture strand, which may be referred to as a “capture oligonucleotide” that is covalently bound to membranes 14. A plurality of oligonucleotide/antibodies conjugates 18 (a-c) are also provided, each of which comprises an antibody 20 (a-c), which may be a primary or secondary antibody, attached to a cleavable transfer oligonucleotide 22 (a-c) which is complimentary to capture strand oligonucleotides 16. A fluorescent-tag 21 may be attached to each oligonucleotide 22. While only a three layer stack 12 and three oligonucleotide/antibodies conjugates 18 are illustrated in FIG. 1 it should be appreciated that substantially more layers and conjugates can be employed depending on the number of targets sought to be identified. Ten, 20, or even 30 or more layers can be employed, for example.

With reference to frames C-E of FIG. 1 use of a preferred embodiment of the present invention to analyze multiple biomarkers in a tissue section is illustrated. Oligonucleotide-antibody conjugates 18 (a-c) are applied to a tissue section 24 that is mounted to a glass slide 26. In the illustration shown in FIG. 1, tissue section 24 has three distinct targets or biomarkers 28 (a-c) of interest although 10, 20, or even 30 or more biomarkers (e.g. cell signaling proteins) can be analyzed using the present invention. After conjugates 18 bind to targets 28 unbound antibodies are washed or otherwise removed from tissue section 24 (not shown). Affinity membrane stack 12 is then applied to tissue section 24. Transfer oligonucleotides 22 (a-c) are then cleaved from conjugate antibodies 20 (a-c) and migrate (upward) through stack 12 (a-c) until transfer oligonucleotides hybridize to their complementary capture strands 16 (a-c) on a particular layer 14 (a-c).

Following transfer and binding of transfer oligonucleotides 22 to the appropriate layer in stack 12 and washing of unbound oligonucleotides, the membranes are separated and scanned or imaged using one of several imaging devices discussed in the sections that follow. The fluorescence intensity per unit area and location on the membrane may then be recorded. These images may then be overlaid upon one another and on a corresponding bright field image of the histochemically stained tissue section using image analysis software and analyzed.

In an alternative embodiment affinity membranes stack 12 may be comprised of generally transparent TEMs enabling them to be imaged together as a stack without the need for separation (FIG. 16) as described in the sections that follow.

It should be readily apparent that while FIG. 1 shows use of the present invention for analysis of tissue sections, affinity membrane stack 12 can be employed for a variety of other applications including use in biosensors to detect pathogens or other environmental analytes.

2. Definitions

The following terms shall have the following meanings as used in this Specification:

“Antibodies” means here in general, any of the following types of analyte-specific reagents: a classical antibody produced in an animal host or a fragment thereof such as a Fab fragment; a variety of recombinant antibody proteins either incorporating or consisting of an antibody fragment or a synthetic ligand selected for its target-binding specificity; synthetic nucleic acid aptamers that performs an analogous function; or in general any kinds of synthetic or semi-synthetic reagents that are engineered or selected to provide an appropriate functionality, namely, to enable the delivery of a signaling oligonucleotide to a specific target molecule in a specimen.

“Antigens” means those target molecules that are used to elicit in vivo, or simulate in vitro, a humoral immune response of an animal, with the intention of obtaining specific antibodies therefrom that recognize one or more of the most characteristic epitopes in that target molecule.

“Capture strand” means an oligonucleotide that is covalently bonded to an assay-specific supportive layer. The capture strand comprises a complementary sequence to the specific transfer strand of an assay in accordance with one embodiment herein.

“Epitopes” means those characteristic portions of an antigenic target molecule to which target-specific antibodies make physical contact in the act of specific binding to that molecule.

“Multiplex” means a capability to perform more than one assay on the same specimen at the same time and, in particular, the use of a set of chemically distinctive physical layers as a substrate upon a set of assays is organized and performed in parallel.

“Oligonucleotides” or “ODNs” means a linear sequence of nucleotides joined by phosphodiester bonds. DNA polymers containing up to 50 nucleotides (or base pairs if double stranded) are generally termed oligonucleotides, and longer polymers are called polynucleotides. “Oligonucleotides” is used synonymously with “polynucleotides” for the present purposes. The oligonucleotides of the invention can range up to a few hundred nucleotides but are generally of a minimum of around 18-25 nucleotides in length if only naturally occurring chemical types are used. The nucleotides can be as short or as long as desired, and they may include protein binding segments such as aptamers, as long as self-hybridization and extrinsic molecular binding activities do not impair the reagent's functionality The oligonucleotides may also comprise peptide nucleic acids (PNAs), locked nucleic acids (LNAs), or other types of chemically modified nucleic acids including a cleavable disulfide bond. The oligonucleotide may be single stranded, or it may incorporate an internal duplex segment that is formed by the hybridization of its own self-complementary sequences. The oligonucleotide also may incorporate fluorescent tags and optional fluorescence quencher units.

“Transfer strand” means an oligonucleotide that is released from an analyte- or target-specific reagent (such as an antibody) that carries information about the position on the specimen surface of the target-specific reagent which is also the position of the target. The transfer strand comprises a complementary sequence to the specific capture strand of an assay.

“Sense” and “antisense” are terms which may be used here solely to distinguish two complementary oligonucleotide sequence tags, without meaning any biological connotations.

“Track-etched membranes” means membranes formed by a process that creates well-defined pores by exposing a dense film to ionizing radiation forming damage tracks. This is followed by etching of the damaged tracks into pores by a strong alkaline solution.

3. Methods of Preparing and Coating the Membranes

a. Membranes

The membranes employed as substrates are preferably “track-etched’ membranes (TEMs). TEMs were invented and patented by General Electric (GE) in the 1960s (see U.S. Pat. No. 3,303,085). Methods of making and using TEMs and are described by Hanot et al. in “Industrial applications of ion track technology,” Nucl. Instrum. Methods Phys. Res. Sect. B, 267: 1019-1022 (2009) and “Expanding the use of track-etched membranes” in IVD Technology November/December/2002 as well as on the Internet websites of GE's Water and Healthcare business units. Examples of membranes that may be employed for use with the present invention include the Isopore™ (polycarbonate film membrane available from Millipore (Bedford, Mass.), the Poretics® polycarbonate or polyester membranes available from Osmonics (Minnetonka, Minn.) or the Cyclopore™ Polycarbonate or Polyester membranes available from Whatman (Clifton, N.J.). Importantly, the etching process results in pores with carboxylic acid residues or other groups that can be covalently bonded to the oligonucleotides as modified herein.

The pore density of the TEM may be between about 10⁷ and 10⁹ but is preferably about 10⁸. The pore size of the TEM may be between about 0.1 μm and 3 μm but is preferably 0.2 or 0.4 μm. The membrane thickness may be between about 5 μm and 20 μm but is preferably about 10 μm. The total surface area including the interiors of the pores may be between about 10 and 50 cm²/cm² of membrane surface but is preferably about 15 cm²/cm².

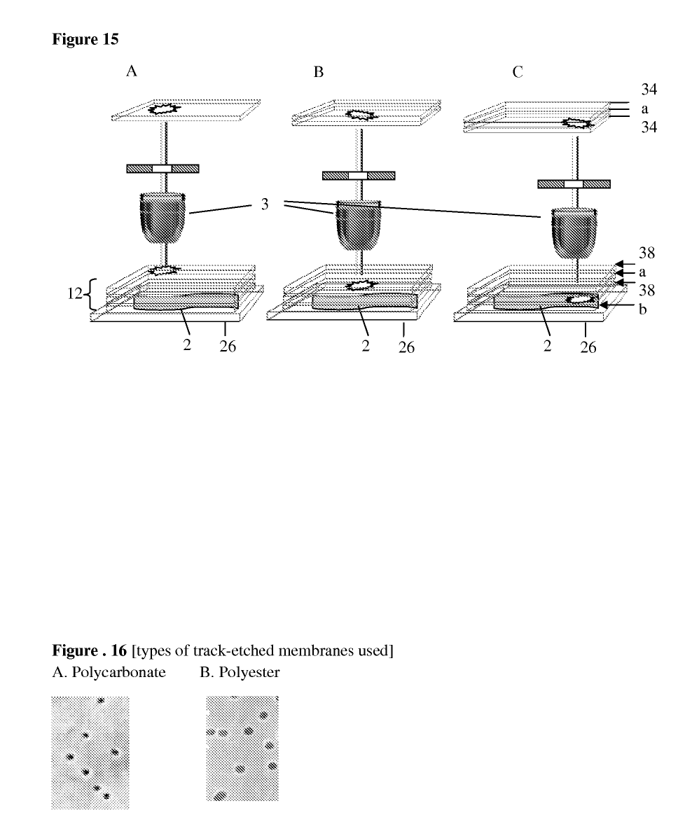

In an alternative embodiment transparent TEMs may be employed in lieu of conventional opaque membranes. A representative transparent membrane that may be used is Cyclopore Polycarbonate Thin Clear Membranes 1.0 μm Pore Size (cat. no. 7091-4710) available from GE Healthcare Whatman (www.whatman.com). As illustrated in FIG. 15 transparent membranes 38 (which could also be hydrogel layers) may be used in conjunction with an imaging device (e.g. a confocal microscope 32) that can optically penetrate a stack of membranes 38 and ascertain the location (3 dimensions) of signals. In FIG. 15 (A) confocal microscope 32 is used to perform an x-y scan of a first layer of the layered sample set 38a, which provides a single two-dimensional image 34a of all transfer molecules captured in that layer. Subsequent scans at advancing perpendicular depths (B & C) provide additional second 34b and third 34c image layers which are aligned with respect to the common x-y plane. Digital image stacks of the data set are subsequently analyzed to quantitate the local signal intensity of each assay target, also serving to allow for orientation of all assays with respect to an image of the target cells (such as tumor cells) present in the same tissue section 36 as obtained after subsequent conventional staining (i.e. with hematoxylin and eosin).

Inert layers may be used to separate some or all assay layers (to facilitate an optical analysis without disassembling the layers), and one or more layers may be used to release reagents that cleave the oligonucleotides from antibodies, or that alter the effective stringency of the oligonucleotide hybridization conditions. The layers may be supported on a firm substrate, which may be both transparent and thin enough so that it does not interfere with a microscopic examination (i.e. less than 120 microns); also the layers may be joined at one or more edges (e.g. by a process of local heating, use of an adhesive or ultrasound, etc.) to facilitate further treatments subsequent to transfer while preserving their alignment.

In another embodiment of the disclosure, the material of the layers can be composed of a hydrogel type of substance such as polyacrylamide or agarose layers. These layers may be transparent, and thus the entire stack of layers can be examined by the use of a confocal microscopy without separation of the layers. The microscopy instrument 32 may be used with transparent hydrogel layers.

b. Oligonucleotides

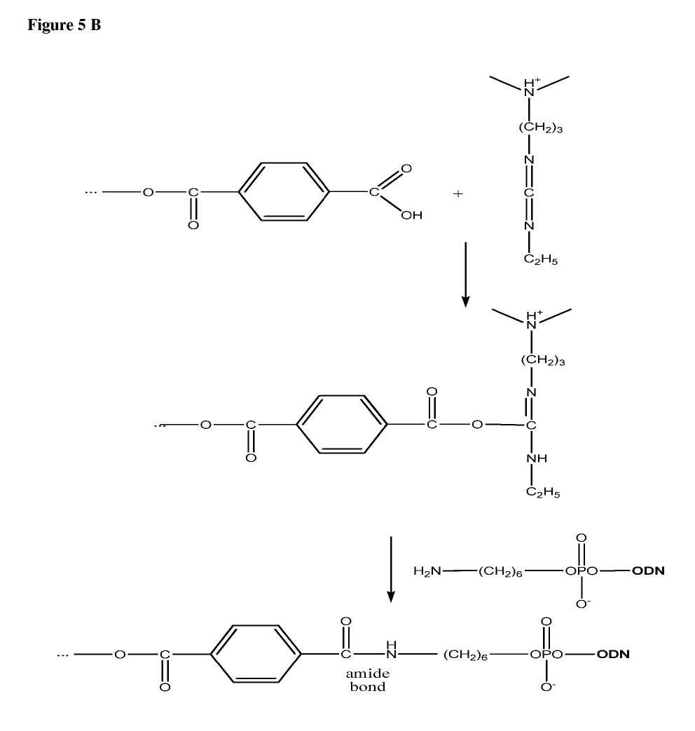

Capture oligonucleotides 16 are preferably linked to one (or more) primary amino groups through a carbon spacer positioned at the 3′ and/or 5′ terminals (FIG. 5). The oligonucleotides are preferably between 18 and 28 nucleotides in length (if composed of natural nucleotides only) and most preferably between about 20-24 nt long with an amino linkage having the formula:

H₂N—(CH₂)ₙ—PO₄—ODN

with n=2-7.

The amino linkage is to either the 3′- or 5′-phosphate end of the oligonucleotide. At the opposite end, some oligonucleotides may also contain a linked fluorophore, Cy5 or fluorescein. Fluorophore-labeled oligonucleotides may be purified by HPLC, or by a standard desalting upon their synthesis. These amino linked oligonucleotides can be ordered from one of many companies or they can be prepared by one who is skilled in the art of oligonucleotide synthesis.

c. Attaching the Oligonucleotides to the Membranes

It is a particular feature of the present invention that the aminated oligonucleotide is attached to the TEM in the presence of a carbodiimide (FIG. 5) or an equivalent condensing agent such as triphenylphosphine/dipyridyl disulfide or triphenylphosphine/carbon tetrachloride To increase the efficiency and quality of the carbodiimide attachment process the TEMs are preferably pre-treated in the following manner.

First, it may be advantageous to de-gas the TEMs. When these membranes are manufactured, about 90 percent of their surface area is comprised of air-filled pores. De-gassing may be accomplished by submerging the membrane in a base buffer, for example, 0.1 M 4-Morpholineethanesulfonic acid (MES) hemisodium salt solution, using a sterile, nuclease free Molecular Biology grade water, and the membrane degassing proceeds under vacuum for 30 min. This step should be repeated, preferably using a fresh salt solution. Alternatively, other solutions may be used for degassing the membrane. These solutions tend to be base solutions that do not adversely affect the membrane. Another buffer that may be used is a 1-methylimidazole buffer.

The buffer solution system is designed to keep the pH constant in the course of a reaction. In the present case, beside the ability to keep pH 5.8-6.2, the buffer must not react with carbodiimide or intermediates it forms with a carboxylic acid. The base pH range may be broader than that presented supra, depending on the conditions and chemicals used. Quoting from “Bioconjugate Techniques,” it should be noted that “other (then MES) buffers may be used as long as they don't contain groups that can participate in the carbodiimide reaction. Generally, it helpful to avoid carboxylate- or amine-containing buffers such as citrate, acetate, glycine, or Tris.” Alternatively, the reaction can be conducted without any buffer by controlling the pH with pH-meter and adding HCl manually.

Other methods of degassing may be used, as long as the integrity of the track etched membrane is kept intact, and provided that the solution used does not interfere with the condensation reaction.

Following membrane degassing and oligonucleotide preparation the TEMs are saturated with the oligonucleotides and given time to adsorb to the degassed track etched membranes. (See Example 1)

After several hours, the amino groups of the aminated oligonucleotides are coupled to the carboxyl groups of the track etched membranes via condensation, and a condensing agent. The preferred condensing agent is a carbodiimide. A number of different carbodiimides may be used for the reaction, including, but not limited to, N-(3-Dimethylaminopropyl)-N′-ethylcarbodiimide hydrochloride, (EDC) (Sigma, Cat. No. E1769). EDC belongs to a class of cold water-soluble carbodiimides. Other cold water carbodiimides that can be used include CMC (1-cyclohexyl-3-(2-morpholinoethyl)carbodiimide and BDC (1-benzyl-3-dimethylaminopropylcarbodiimide). Other non-carbodiimide condensing agents that may be used include oxidants such as triphenylphosphine or a reductant such as dipyridyl disulfide or carbon tetrachloride, preferably used in an organic solvent.

The period of incubation of the track etched membrane, the aminated oligonucleotides, and the condensing agent can range from six to 14 hours. The number of hours for incubation may vary, as determined by the thickness of the membrane, the type of track etched membrane used, and other variables.

While the condensation reaction is quite thorough, there is the possibility that unreacted aminated oligonucleotides may remain adsorbed to the membrane. This is problematic because when the transfer oligonucleotides 22 pass through the stack they could bind nonspecifically to the wrong layer if their complementary strands are not rigidly bound to their assigned layer. Thus, it is advantageous to remove the adsorbed oligonucleotides by washing them until all or virtually all of the adsorbed oligonucleotides are removed from the track etched membranes, thereby leaving behind only those oligonucleotides covalently bonded to the track etched membrane. To accomplish, this, a rigorous washing is necessary that does not chemically adversely affect the covalently bound oligonucleotides nor the track etched membranes to which the oligonucleotides are bound. Preferably a solvent, such as a polar aprotic solvent, is used. In one embodiment, acetonitrile is used. The acetonitrile can reside in water, or in a phosphate solution. Similarly, the track etched membranes, can be washed in a phosphate solution before and/or after the washing in the acetonitrile solution. It is also advantageous to saturate the membranes with the aminated oligonucleotide prior to exposure and treatment with the condensing agent. This is accomplished by adding the oligonucleotide solution to the buffer solution in which the membranes were degassed, after which the solution containing the condensing agent is added.

4. Antibody/Oligonucleotide Conjugates

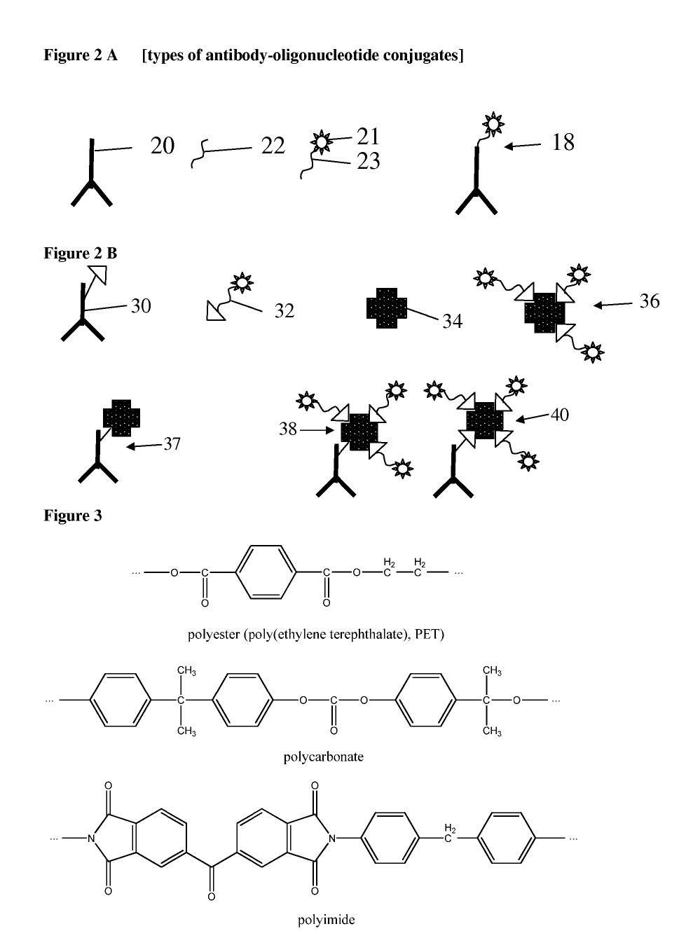

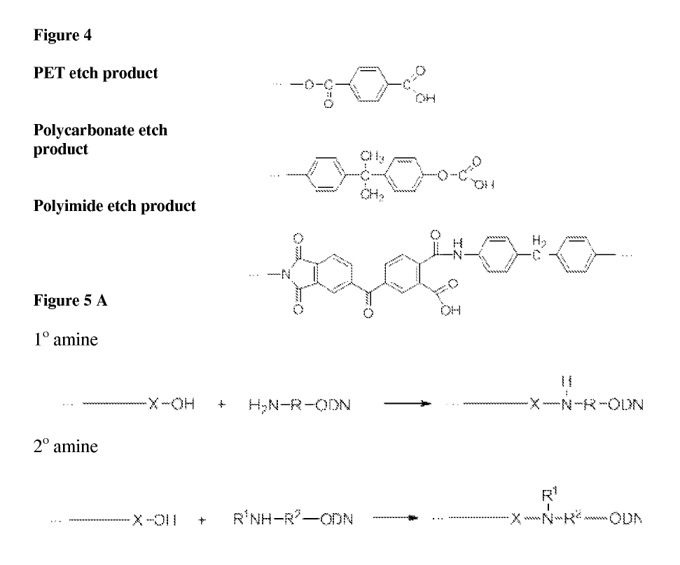

With reference to FIGS. 1 and 2, antibody/oligonucleotide (“Ab/oligo”) conjugates 18, 38 or 40 are designed to work with the affinity TEMs 14 by detecting the targets 28 in the tissue sample 24 and releasing a fluorescently tagged transfer oligonucleotide 23, 32 or 36 to bind to a corresponding layer 14 in stack 12 containing capture oligonucleotide 16. A preferred assay specific ligand is an oligonucleotide transfer strand, such that, as in the figure, a specific oligonucleotide transfer strand is attached to a specific antibody, which is in turn is uniquely attached to a specific antigen when applied to tissue 24.

In a preferred embodiment, the oligonucleotide is linked to an antibody domain that is not directly involved in binding of the antibody to its target antigen. Secondary antibody conjugates may be used to detect unconjugated primary antibodies, provided that for the use of multiple secondary antibodies in multiplex mode, an immunological difference between the specificity of the different secondary antibodies would be required (e.g. anti-rabbit vs. anti-mouse). In another embodiment, a secondary antibody can be complexed with a primary antibody before the primary is applied to the tissue section.

FIG. 2A illustrates a method in which the transfer strand 22 is linked to the antibody 20 through a direct covalent bond or a series of such bonds; FIG. 2B exemplifies a method in which there is a noncovalent linkage of the transfer strand 32 to the antibody 20, as exemplified by a biotin-streptavidin noncovalent bond in conjugates 38 and 40.

The link between antibody and oligonucleotide may be through an intermediate molecule such as streptavidin. In one embodiment the antibody 20 is linked to streptavidin 34 to form intermediate conjugate 37 by performing a reaction using a covalent bifunctional crosslinker. Where the bifunctional crosslinker may contain a labile bond that as previously described, which bond may be cleaved by a reducing agent or other means recognized by a person of ordinary skill in the art. Biotinylated oligonucleotides 32 are reacted with this to form complex 38. In a related embodiment a biotinylated primary antibody 30 is used. Biotinylated oligonucleotides 32 are bound to the tetravalent molecule streptavidin to form conjugate 36. The biotinylated oligonucleotides 32 may contain a labile bond, which may be cleaved by a reducing agent or other means recognized by a person having ordinary skill in the art. Conjugate 36 is then reacted with biotinylated antibody 30 to form a complex 40. The link between the antibody and the transfer oligonucleotide preferably may be provided with a labile bond, which may be either covalent or noncovalent. This bond allows the transfer oligonucleotide to be decoupled from the antibody (or intermediary antibody-binding ligand) so as to solubilize the oligonucleotide separately from the tissue for transfer to the capture strand in a layer. Various chemicals and processes for breaking the antibody-oligonucleotide bond can be used; for example, beta-mercaptoethanol can be used to break a disulfide bond; or one could dissociate a duplex consisting of a linker strand that is covalently linked to the antibody and a complementary transfer strand by increasing the temperature to exceeded the melting temperature of that duplex by at about 10-20° C. Interactions of oligonucleotide-conjugated primary antibodies with tissue and of released transfer oligonucleotides with capture strand-coated membranes is illustrated in FIG. 1.

The migration of the transfer oligonucleotide may be facilitated by cleavage of the bond that attaches the transfer oligonucleotide to the antibody. There are a number of ways known in the art to break the bond, which are surveyed in Bioconjugate Techniques, Second Edition, by G. T. Hermanson, Elsevier 2008 which is incorporated herein by reference in its entirety). In one embodiment, the bond may be broken by gentle heating (37-55° C.) after the stack has been applied to the bound sample, i.e. during the transfer.

In another embodiment, an ancillary chemical, which could be a stabilized reducing agent such as beta-mercaptoethanol or TCEP (Thermo Pierce cat. no. PI-77720, the manual for which is herein incorporated by reference) is pre-positioned in the layers at a working concentration therein, and upon contact of the outermost layer with the specimen, reacts with the crosslinker, causing rapid and complete cleavage of the crosslinker structure between the antibody and the tagged oligonucleotide within 1-60 minutes.

Alternatively, other linkage structures could be used to enable solubilization of the oligonucleotide from the antibody. Such linkages include but are not limited to a photosensitive linkage, or an oligonucleotide subsequence that could bind a chemical such as an enzyme that would cause localized cleavage within the oligonucleotide. There are numerous chemical structures that would allow for an easily breakable bond between the oligonucleotide and the antibody. Displacement by competitive binding of another oligonucleotide could be used to release the assay-specific ligand.

In the process, one type of specific capture molecule (the capture oligonucleotide strand) is attached to each of said vertically ordered, bioaffinity layers. The assay-specific ligands (transfer oligonucleotides) that are attached to the primary or secondary antibodies (which here exemplify the class of analyte-specific ligands) are released from the tissue sample (by application of a chemical or by mild heating to 37-55° C.), and they necessarily diffuse away from the specimen, along a direction of movement which is determined by the vector of their chemical concentration gradient. Each of the assay specific ligands (e.g. a transfer oligonucleotide) passes through the stack until it meets a bioaffinity ligand (the capture oligonucleotide which is to it the antisense strand) to which it specifically binds according to the pertinent type of bioaffinity chemistry (e.g. formation of duplex DNA). It should be noted that in some cases the antibody, still attached to the transfer strand, migrates with the transfer strand, with no adverse effects to the reading of the results.

If a secondary antibody rather than a primary antibody is conjugated with an oligonucleotide, normal care must be taken to avoid cross reactions with different primary antibodies. Either those primary antibodies must be distinguishable by their respective secondary antibodies, or a complex of a primary antibody and monovalent secondary antibody fragment could be formed prior to application of the mixed antibody complexes to the tissue specimen (not illustrated).

A fluorescent tag 21 may be attached to transfer oligonucleotide 22 for subsequent detection by an imaging instrument such as fluorescent scanner the Typhoon® scanner available from GE Life Sciences. Examples of fluorescent tags that may be used include dyes such Cy5, Cy3, fluorescein, etc. Fluorescent tag 21 is preferably added to the oligonucleotide during stepwise synthesis in a manner commonly known in the art. Whereas in the examples provided herein a single dye was employed it should be appreciated that different colors can be employed which may be advantageous when using the transparent membrane approach (FIG. 15). In lieu of a fluorescent tag various methods may be employed to detect duplexes of the oligonucleotides. A double-strand specific DNA binding dye may be used analogously to a real time PCR detection method, such as SYBR Green I available from Invitrogen, Inc. (Carlsbad, Calif.).

5. Uses and Applications of Coated Membranes and Conjugates

Oligonucleotide coated affinity membranes 12 and Ab/oligo conjugates 18 may be used in a variety of configurations for a variety testing purposes. One important application, illustrated in FIG. 1 and described in Examples 8-10, involves the profiling of multiple biomarkers in a tumor section. For this application a tumor is sectioned and prepared according to standard clinical pathology processes for routine immunohistochemistry (IHC) analysis (‘special staining’) except that Ab/oligo. conjugates 18 are used in lieu of standard IHC antibodies. (It should be appreciated that virtually any IHC antibody can be conjugated with an oligonucleotide according to the processes described herein.)

Membranes 12 or 14 are then dipped into an oligonucleotide binding buffer containing a reducing/releasing agent, such as one that can cleave the disulfide bonds in the Antibody/oligonucleotide conjugates 18 such as TCEP or beta-mercaptoethanol. The membranes are assembled into a stack 12 in a chosen order as to the identity of the oligonucleotides coated on them, which ordering serves to spatially organize the assays which are to be performed.

Following application and binding of conjugates 18 to tissue section 24 the tissue sample is rinsed with binding buffer without a reducing/releasing agent, and membrane stack 12 is placed directly on top of the tissue section. The excess buffer volume is expressed from the stack by enclosing it between protective layers and applying gentle pressure. In a preferred embodiment, the stack is first assembled and then applied to a specimen surface where a remnant of wash buffer persists from the last specimen wash step. The excess wash buffer is then expressed from the specimen surface by applying a weight or mechanical pressure to the entire assembly. Both the tissue section and membrane stack are preferably enveloped in a fluid impermeable enclosure (e.g. plastic bag) and kept moist throughout the transfer process. When membrane stack 12 is applied to the tissue specimen a weight or similar pressuring device such as spring metal clips (not shown) may be applied to express an excess buffer volume and assure uniform contact with the tissue specimen throughout the transfer process. The enclosed stack and slide mounted tissue section are then incubated for an optimal time (between about 10 minutes and 4 hours) and a temperature range of between room temperature and 70° C. depending on the requirements of the kinetics of the chemical reactions which are being performed; such requirements could include the concentration of a reducing agent, the diffusion rate of a transfer strand through the type of substance used to form the layered supports, etc.

In an embodiment in which a transfer strand is linked to an antibody through a disulfide bond, the reducing agent functions to cleave the transfer oligonucleotide strands 22 from antibody 20, or transfer strands 32 from complex 38 or 40, or complex 36 from antibody 30. The transfer strands then diffuse upward through the porous membrane stack and bind to the membrane layer 14 supporting the complimentary oligonucleotide strand as shown in FIG. 1 E. Examples 5, 8, 9 and 10 exemplify the use of this method.

Membrane stack 12 is then removed from the enclosure and washed in a buffer. In one embodiment (opaque or translucent membranes) the membranes layers are separated from one another, dried, and arrayed on a flat bed fluorescence scanner (not shown). The scanner then records the location and intensity of fluorescent tags 21 on each membrane. This data can be analyzed using a variety of software programs such as ImageQuant (Molecular Dynamics) to permit the user to quantitate and correlate the assayed multiple biomarker expression levels at given locations within the tissue section.

A fluorescence quencher can be used in the transfer strand to suppress light emission of the fluorescent tag until hybridization to the capture strand occurs on the layer (e.g. FRET); useful fluorescence tags and fluorophore-quencher pairs can be used that are well known to one skilled in the art of fluorescence detection methods (these principles are outlined in the short report, “Fluorescence and Fluorescence Applications”, 2005, available online from Integrated DNA Technologies, incorporated herein by reference in its entirety). The membrane stack may be dismantled such that each membrane is read separately, or the entire stack may be read by use of a confocal microscope if the individual membranes are transparent or translucent.

In some embodiments, an antibody may be provided with a covalently conjugated linker strand that is truncated and forms a low-affinity duplex with a transfer strand; the transfer strand may be subjected to thermal dissociation from the antibody during the transfer step by mild heating (e.g. by 15-30° C.). In general, persons who are proficient in the art would recognize many ways to cause dissociation of various types of transfer complexes on the specimen, while providing for the subsequent binding of the transfer molecule to the capture layer.

In some embodiments, an arbitrary number of oligonucleotide sequence tags (for example, U.S. Pat. No. 5,635,400), sometimes referred to as bio-barcodes, are identified that do not cross-hybridize when mixed in solution. In particular, the transfer oligonucleotides do not cross-hybridize to one another, and they do not cross-hybridize to the capture oligonucleotides of the other transfer oligonucleotides that are attached to the capture layers. These embodiments enable modular expansion of the number of assays per run and a replicable assay development procedure all based on the universality of the oligonucleotide transfer/capture system as here employed. Similar universal multiplex assays have been developed as biochemical techniques, for example these have been used in microarray analysis of PCR products (Favis, R. et al (2000) Universal DNA array detection of small insertions and deletions in BRCA1 and. BRCA2. Nat. Biotechnol. 18, 561-564).

In addition to use with tissue sections, oligonucleotide coated affinity membranes 12 and Ab/oligo conjugates 18 may be used in other testing formats, for example, assays of blood or other bodily fluids in a multi-well plate format in the same manner as that described for the Layered Peptide Arrays as described by Emmert-Buck, et al. in U.S. Patent Application Pub. No. 2009/0215073 A1 (application Ser. No. 12/289,736) and in Clinica Chimica Acta 376 (2007) 9-16 and the Journal of Molecular Diagnostics, Vol. 9, No. 3, July 2007 pgs. 297-304.

Affinity membranes 12 may also be used without conjugates 18, for example to detect DNA or RNA targets in blood or an environmental sample (soil, air, or water) as a component of biosensors. Examples of use of TEMs for various biosensors and diagnostics for which the present invention may be employed are described by Hanot et al. in “Industrial applications of ion track technology,” Nucl. Instrum. Methods Phys. Res. Sect. B, 267: 1019-1022 (2009) and by Jones et al. in “Expanding the use of track-etched membranes” in IVD Technology November/December/2002.

### EXAMPLES

Introduction to Examples. The covalent immobilization of single-stranded DNA was demonstrated on commercially available track-etched membranes using oligodeoxynucleotides 20-24 nt long. In the Examples below the membranes were mainly circular discs with a diameter of 6.5 mm that fits the wells of a standard 96 well plate. Before the use, the membranes were submerged into a conjugation buffer and degassed under vacuum with two changes of the buffer. Quantitative and semi-quantitative description of the immobilization is based on the use of fluorescently labeled oligonucleotides and measurement of the fluorescence intensity in reaction mixtures, washings, and on membranes. The measurements and image acquisitions were done on 1420 Multilabel Counter Victor² (Wallac, Turku, Finland) and Storm 860 Gel and Blot Imaging System or Typhoon Trio+ Variable Mode Imager (Amersham Biosciences, Sunnyvale, Calif.). The images were treated with the Molecular Dynamics software ImageQuant, version 5.2.

Membranes, DNA, and Reagents. The track-etched membranes in a sheet format or circular were from two sources: GE Water & Process Technologies (Trevose, Pa.) and the Belgian company it4ip (Seneffe, Belgium). Specifications of the membranes are presented in TABLE 1. The majority of oligodeoxynucleotides was synthesized by Eurofin MWG Operon (Huntsville, Ala.) and some were obtained from Integrated DNA Technologies (Coralville, Iowa). Structures of the oligonucleotides and ways of their purification are shown in TABLE 2. Other principal reagents were N-(3-dimethylaminopropyl)-N′-ethylcarbodiimide (EDC) hydrochloride, 4-morpholineethanesulfonic acid (MES) hemisodium salt, acetonitrile, HPLC grade, and 0.2 M phosphate buffer, pH ˜7.4, containing 2.98 M NaCl and 0.02 M EDTA (SSPE buffer 20× concentrate) (Sigma-Aldrich Corp., St. Louis, Mo.). The oligonucleotide solutions were prepared using DEPC-treated, sterile nuclease-free water (EMD Chemicals, Gibbstown, N.J.). Milli-Q water passed through the BioPak ultrafilter (Millipore, Billerica, Mass.) was used to prepare other solutions and buffers.

TABLE 1

| Membrane | Pore size, | Pore Density, | Thickness, | |
|---|---|---|---|---|
| & Cat. No. | μm | cm⁻² | μm | Vendor |
| Polycarbonate | 0.4 | 1 × 10⁸ | 10 | GE |
| K04SH02500 | | | | |
| K04SH81030 | | | | |
| K04CP02500* | | | | |
| Polyester | 0.4 | 1 × 10⁸ | 10 | GE |
| T04CP02500 | | | | |
| Polycarbonate | 0.4 | 1.6 × 10⁸ | 10 | it4ip |
| 1000M10/811N400/A4 | | | | |
| Poly(ethylene | 0.4 | 1.0 × 10⁸ | 12 | it4ip |
| terephthalate) | | | | |
| 2000M12/811N400/A4 | | | | |
| Polyimide | 0.4 | 2.0 × 10⁸ | 12 | it4ip |
| 3000M12/821N400/A4 | | | | |

*The membranes are coated with a wetting agent, poly(vinylpyrrolidone).

TABLE 2

| Name | Structure | hybridizes | Purity | Vendor |
|---|---|---|---|---|
| ATP17 | [Cy5]TGAT₂GTAGTATGTAT₂GATA₃G[AmC7] | ATP95 | HPLC | Operon |
| ATP95 | [Fl]CT₃ATCA₂TACATACTACA₂TCA | ATP17 | HPLC | Operon |
| ATP60 | [AmC6]TCAT₃AC₂A₂T₃AC₂A₂T | ATP25 | desalting | IDT |
| ATP25 | [Cy5-Sp9]AT₂G₂TA₃T₂G₂TA₃TGA | ATP60 | PAGE | Operon |
| ATP73 | [Cy5]AT₂G₂TA₃T₂G₂TA₃TGA | ATP60 | HPLC | Operon |
| | [ThiSS][BioTEG] | | | |
| ATP61 | [AmC6]CT₃CT₃CT₃CT₃CT₃ | ATP72 | desalting | Operon |
| ATP72 | [Cy3]AAAGAAAGAAAGAAAGAAAG[AmC7-Q] | ATP61 | HPLC | Operon |
| ATP62 | [AmC6]CTACTATACATCT₂ACTATACT | ATP74 | desalting | Operon |
| ATP74 | [Cy5]AGTATAGTAAGATGTATAGTAG | ATP62 | HPLC | Operon |
| | [ThiSS][BioTEG-Q] | | | |
| ATP80 | [AminoC6]AAATCATCAATCACTTTAAT | ATP76 | desalting | Operon |
| ATP76 | [Cy5]ATTAAAGTGATTGATGATTT | ATP80 | HPLC | Operon |
| | [ThiSS][BioTEG-Q] | | | |
| ATP82 | [AminoC6]CTACAAACAAACAAACATTAT | ATP77 | desalting | Operon |
| ATP77 | [Cy5]ATAATGTTTGTTTGTTTGTAG | ATP82 | HPLC | Operon |
| | [ThiSS][BioTEG-Q] | | | |

| Modification abbreviation | chemical |
|---|---|
| [Cy5] | 5′ CY 5 fluorescent dye |
| [Fl] | 5′ Fluorescein fluorescent dye |
| [AmC6] | 5′ primary amine with 6 atom spacer |
| [AmC7] | 3′ primary amine with 7 atom spacer |
| [Cy5-Sp9] | 5′ CY5 with 9 atom spacer |
| [ThiSS] | Dithiol (disulfide), internal |
| [BioTEG] | Biotin with 22 atom spacer |

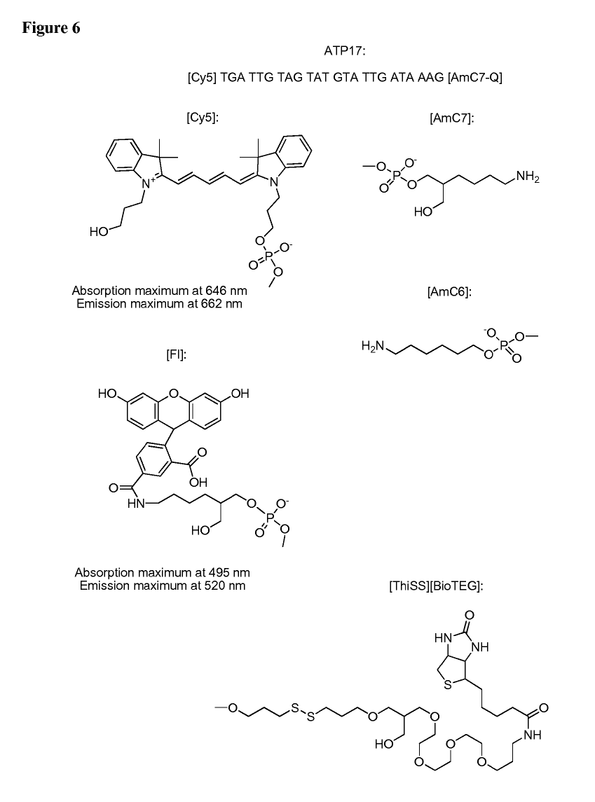

Structures of the modifications (Cy5, AmC6, etc.) are shown in FIG. 6.

### Example 1

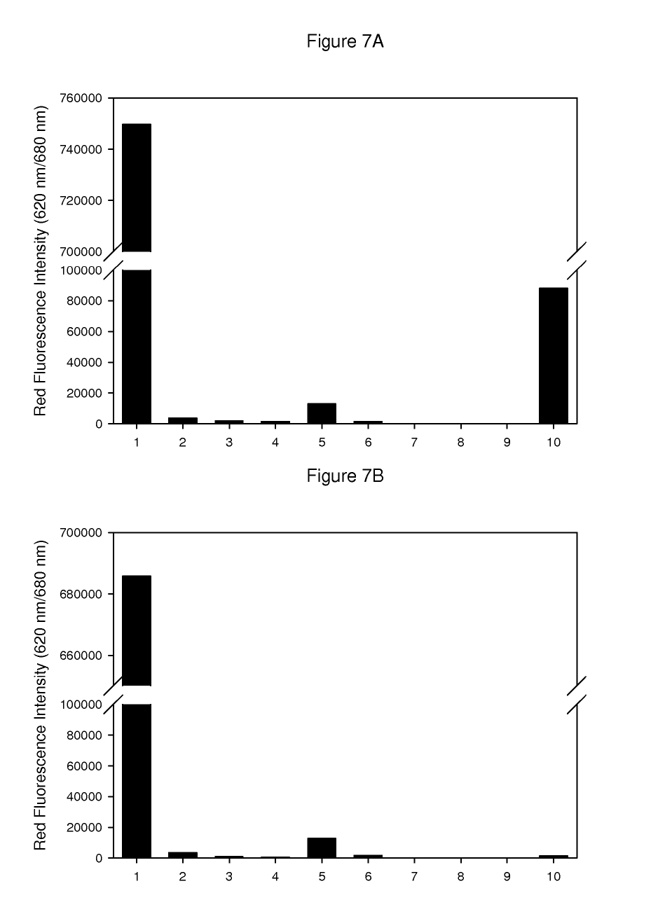

Covalent coating of TEMs via their joint incubation with aminated DNA and carbodiimide. Pairs of the membrane discs degassed in 0.1 M MES buffer, pH 6.1 were submerged without overlapping in 500-μl vials in 80 μl of 62.5 μM aminated ODN in 0.125 M MES buffer and kept there at a room temperature light-protected if the ODN had also an attached fluorophore. Upon 2 hour incubation vials received 20 μl of the freshly prepared 0.5 M EDC in water while vials with control membranes received just water. After the overnight reaction the incubation mixtures were withdrawn and the membranes were washed with 100 μl of 6×SSPE buffer (thrice, each time for 30 min) and then with 100 μl of 10% acetonitrile (thrice, each time for 30 min). The coated membranes were stored in 6×SSPE buffer at 4-6° C. For analytical purposes, the incubation mixtures, washings as well as the resulting membranes (covered with 100 μl of 6×SSPE buffer) were placed in designated wells of 96 well plates and fluorescence intensity was recorded usually at 0.1 s exposures. Such acquired data are presented in FIGS. 7-9.

FIG. 7 describes results of the ATP17 covalent binding to a track-etched polyester membrane. In the procedure used, the membranes were first saturated with the ODN in a prolonged incubation and only then the immobilization was initiated by the carbodiimide addition. By comparing bars 1 and 10 in FIG. 7A, one might conclude that about 10% of the ODN appears covalently bound to the membrane. When passed through the same treatment but without carbodiimide, the membrane retains less than 0.3% of the ODN applied (compare bars 1 and 10 in FIG. 7B). Although polyester (poly(ethylene terephthalate) or PET) is considered as a naturally hydrophilic polymer, data in FIG. 7 show that three consecutive washings with a phosphate buffer (bars 2-4) are less effective in deleting the non-covalently bound ODN from the polyester membrane than single washing with 10% acetonitrile (bars 4 in FIGS. 7A&B).

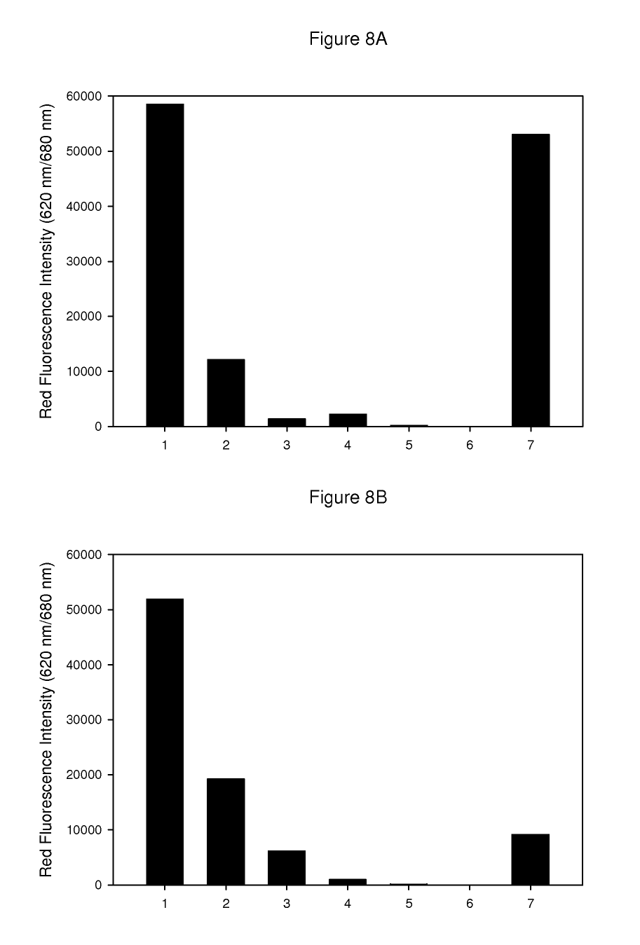

The ATP17 covalent binding to track-etched polycarbonate membranes is characterized in FIG. 8. In contradistinction to polyester, polycarbonate is a highly hydrophobic polymer and its film etching should presumably result not in carboxylic acid end groups but carbonic acid end groups. Still, the procedure provides of the polycarbonate membrane coating (see bar 7 in FIG. 8A) comparable with that achieved with polyester membranes (bar 10 in FIG. 7A). Bars 1 in FIGS. 8A&B also show that upon completion of the treatment with or without the use of carbodiimide and regular washings the membranes retain almost the same amount of the ODN. Yet, in the subsequent washings with 10% (bars 2 and 3) and 30% (bars 4 and 5) acetonitrile, the membranes treated without carbodiimide lose nearly 83% of the ODN while the counterparts still retain the most of it (compare bars 7 in FIGS. 8A&B).

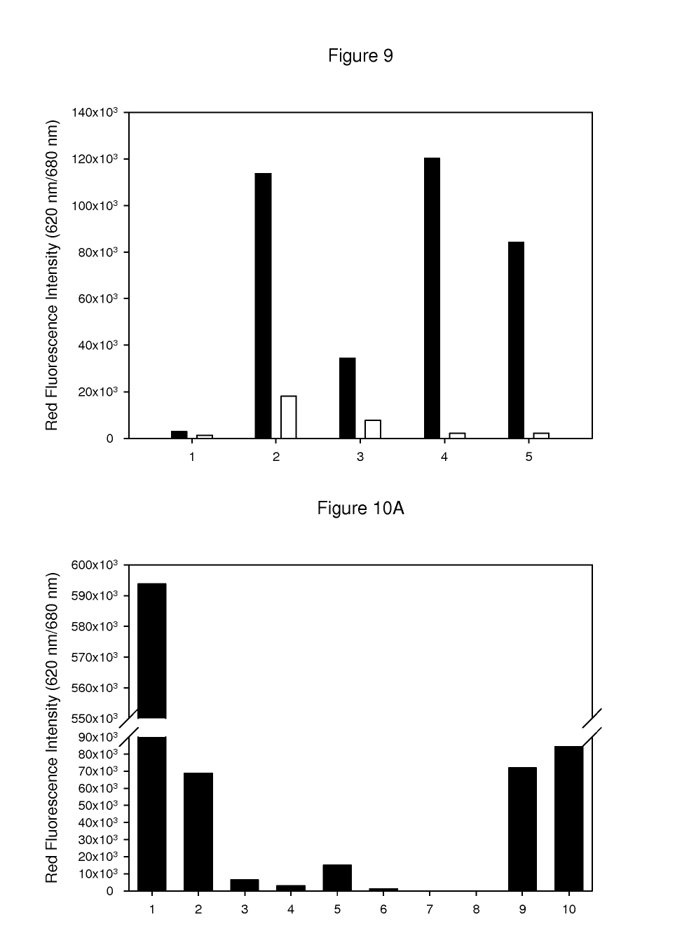

Data in FIG. 9 are to confirm that the procedure of ODN covalent binding to commercially available TEMs works well irrespectively who manufactured them. As seen in particular, the use of polyester membranes obtained from Belgium company it4ip (bar 2) and GE Water & Process Technologies (bar 4) resulted in their nearly identical coating with ATP17. Considering the intensity of the signals from the bound ODN (gray bars in FIG. 9), it can be concluded that on average the polyester, polycarbonate, and polyimide membranes provide correspondingly high, medium, and small binding capacity. Importantly, that the background signal caused by the ODN binding independent on carbodiimide decreases in the inverse relation; this follows from the comparison of the gray and white bars in FIG. 9.

### Example 2

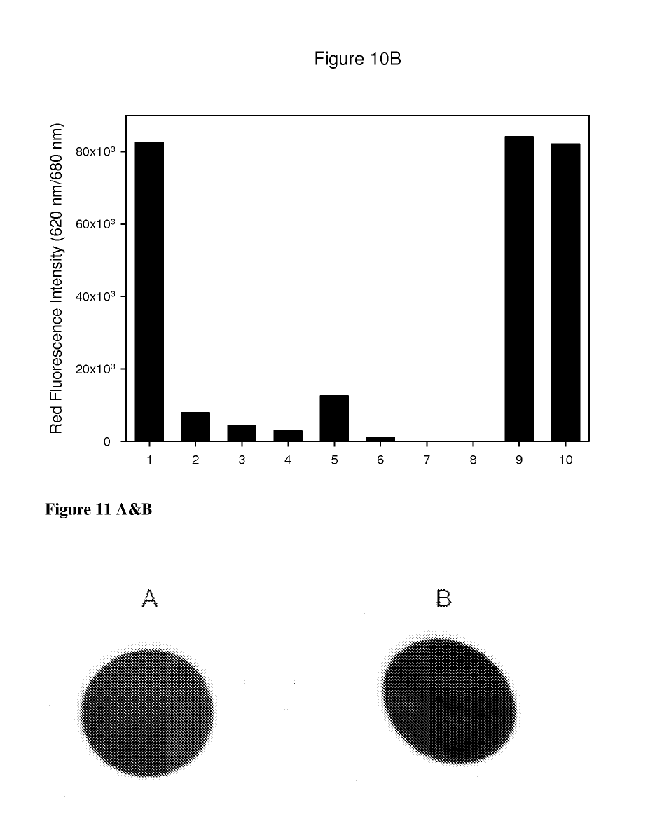

Covalent coating of TEMs via their sequential incubation with aminated DNA and carbodiimide. Pairs of the membrane discs degassed in 0.1 M MES buffer, pH 6.1 were submerged without overlapping in 500-μl vials in 100 μl of 50 μM aminated ODN (ATP17) in 0.1 M MES buffer and kept there at a room temperature light-protected if the ODN had also an attached fluorophore. Upon 4 hour incubation the membranes were transferred into the vials with 100 μl of the freshly prepared 0.1 M EDC in 0.1 M MES buffer and the incubation continued overnight. Washing of the membranes was done as described in Example 1. The comparison of the two ways of the coating is shown in FIG. 10. This EXAMPLE is to show that the covalent coating of TEMs via carbodiimide condensation could be the same if instead of adding carbodiimide in an ODN solution with a membrane this membrane would be taken out of the solution upon its saturation and placed in another, containing only carbodiimide solution. In such a case, the ODN solution would not be contaminated with added carbodiimide and it could be used multiple times. Based on the comparison of bars 9 and 10 (which refer to pairs of membranes treated in parallel) in FIGS. 10A&B it follows that both variants of the coating lead to practically identical products.

### Example 3

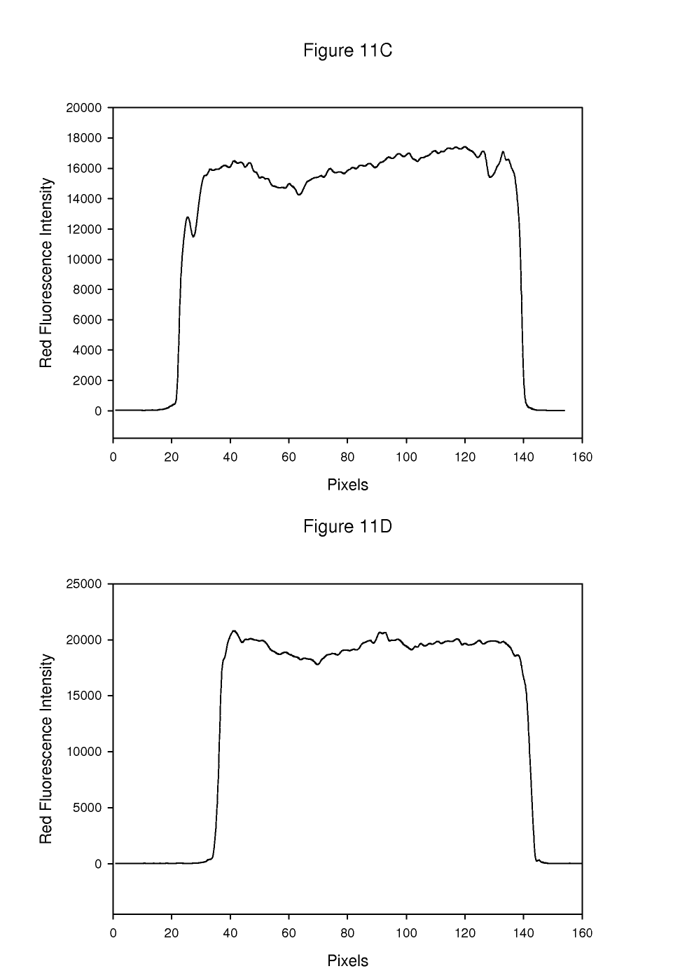

Demonstration of the evenness of the covalent coating. The membrane disks coated with the aminated Cy5-labeled oligonucleotide as in EXAMPLES 1 and 2 and then dried between sheets of Whatman filter paper were placed on the Typhoon Trio+ platen, covered with a regular glass slide and their images were acquired at a normal sensitivity. To avoid saturation of the signal, excitation was done with a green laser (532 nm) instead of the matching red laser (633 nm), emission was registered at 670 nm at a pixel size of 50. Thus obtained fluorescent images of the ATP17-coated polycarbonate membranes are shown in FIGS. 11A&B.

Distribution of the oligonucleotide along diameters of the coated disks was revealed with the ImageQuant software and shown in FIGS. 11 C & D. As seen, both variants of the ODN covalent binding result in a fairly even coating: the joint incubation provided variations within ±15% (FIG. 11C) and the sequential incubation gave even better result, ±5% (FIG. 11D).

### Example 4

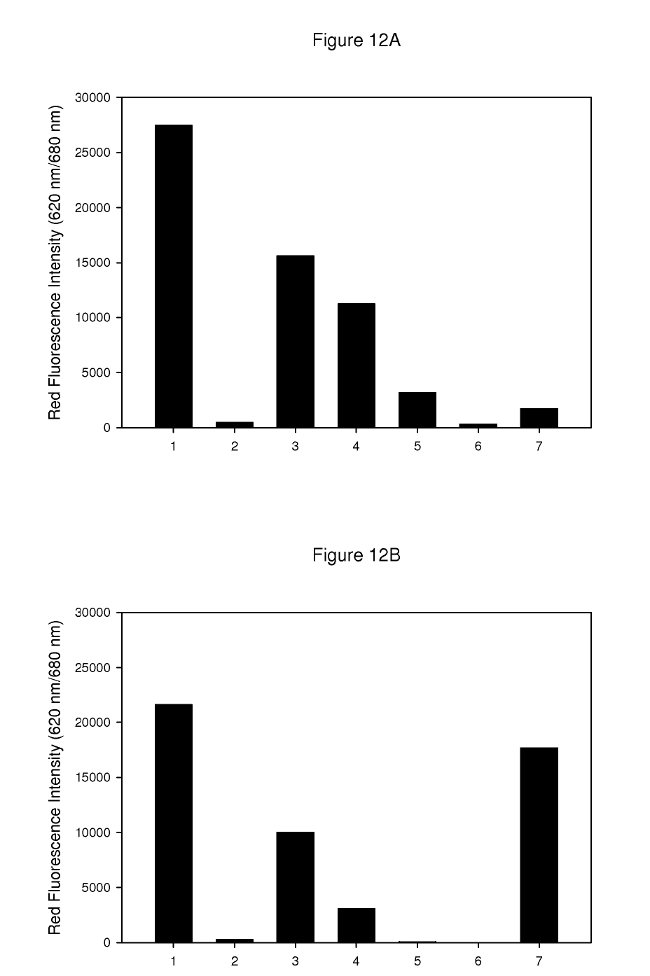

Hybridization and thermal dissociation on a track-etched membrane. Pair of membrane disks coated with ATP60 as in EXAMPLE 1 and washed with 6×SSPE buffer was incubated at a room temperature for 2 hours in 100 μl of 10 μM complementary Cy5-labeled ATP25 in the buffer. Another pair of plain membrane disks was kept in the identical solution to serve as a control. Upon four 30 min-washings with the buffer the disks were transferred into designated wells of a 96 well plate and intensity of their red fluorescence was measured. The disks were further washed with 10% acetonitrile (thrice), 30% acetonitrile (once), and 6×SSPE buffer. The volume of each washing was 100 μl and intensity of the red fluorescence in the washings and resulting membranes is shown in FIG. 12. The data show that an oligonucleotide (ATP60) covalently bound to polycarbonate membrane preserves its ability to hybridize with a complementary oligonucleotide (ATP25). Although ATP25 can interact with the plain membrane non-specifically (FIG. 12A), 93.5% of it can be washed out by the employed washings (compare bars 1 and 7 in FIG. 12A). The same washings leave 83% of ATP25 on the membrane coated with ATP60 (FIG. 12B, compare bars 1 and 7).

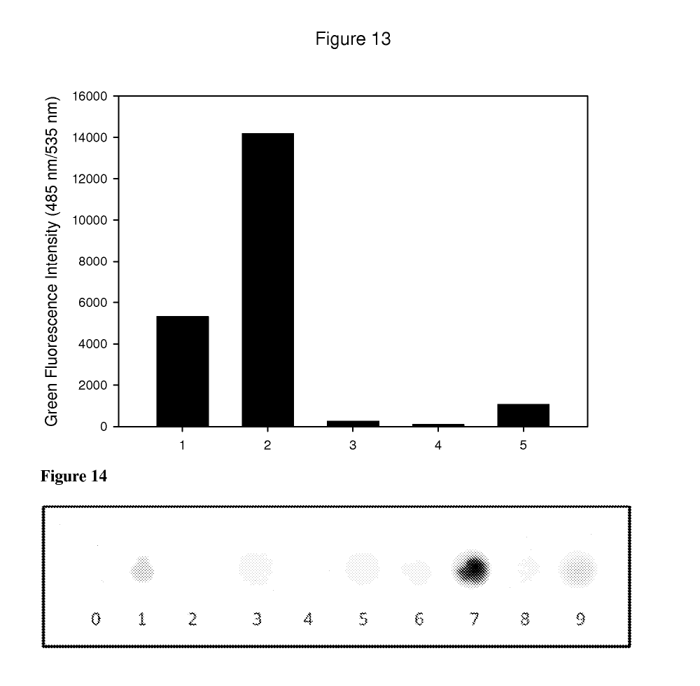

Another pair of membrane disks coated with ATP17 and washed as in EXAMPLE 1 was incubated at 50° C. for 10 min in 100 μl of 25 μM complementary fluorescein-labeled ATP95 in 6×SSPE buffer. This was followed by the 3 hr annealing to a room temperature and washings with the buffer. Upon recording the green fluorescence intensity retained by the disks they were placed into a vial with 100 μl of 25 mM phosphate buffer, pH 7 and heated up to 60° C. In 15 min the solution from the vial was withdrawn and the heat extraction was performed two times more. Intensity of the green fluorescence in the discs before and after thermal dissociation as well as in the hot extracts is shown in FIG. 13.

It is supposed that a duplex formed in solution by two complementary oligonucleotides can be destroyed (melted) at an elevated temperature. Data in FIG. 13 demonstrate that this is also happening when one of the two is immobilized on a track-etched polycarbonate membrane. The pair of ATP17 and ATP95 has an estimated melting temperature of 54° C. Bars 2-4 in FIG. 13 relate to amount of ATP95 released from the immobilized duplex into solution at 60° C. while bar 10 shows what remains (less than 7%) on the membrane after the thermal dissociation.

### Example 5

Hybridization in a stack of track-etched membranes of an oligonucleotide released from an antibody conjugate bound to an antigen. The complex of an antigen with an antibody-streptavidin conjugate and bound biotin-modified ATP73 was prepared on the irregularly shaped piece of a polycarbonate membrane as follows. First, the membrane was coated with rabbit IgG and then blocked with 1% BSA. Second, the membrane was incubated with a conjugate of the goat anti-rabbit antibody and streptavidin that retained ATP73. Upon washing with 4×SSC buffer contained 0.1% SDS the membrane was placed on a supporting polycarbonate PVP-coated disc and covered by a stack in that PVP-coated discs separated the discs with ATP62, ATP61, and ATP60 immobilized as in EXAMPLE 2. The concerted release of the ATP73 fragment and its hybridization with ATP60 were induced by placing on the stack's top a filter paper disc wetted with 4×SSC buffer contained 0.1% SDS and 5 mM Tris[2-carboxyethyl]phosphine hydrochloride (TCEP). After keeping the stack at 45° C. for one hour it was disassembled and the discs were washed thrice with 1×SSC buffer contained 0.25% SDS at 47° C. Fluorescent images of the discs were registered using Storm 860 scanner (FIG. 14). Intensity of the red fluorescence associated with each disc in the stack was also quantified using the microplate reader.

### Example 8

### Identification of Multiple Biomarkers from Breast Tumor Sections and Comparison with Immunofluorescence Histochemistry

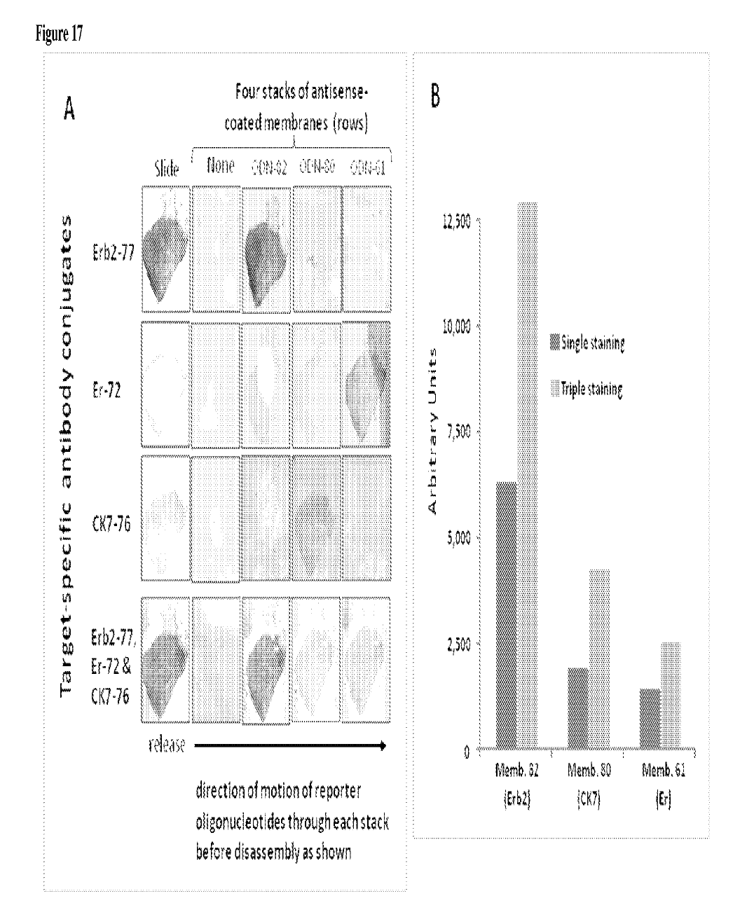

Breast cancer pathology specimens in paraffin blocks were purchased from a commercial tissue bank (Asterand, Inc. or Bioserve, Inc.), 5 μm sections were cut and prepared for staining with conventional immunohistochemistry sample preparation methods. Membranes were prepared by conjugation with oligonucleotides (ODNs) ATP61, ATP80 and ATP82 by the methods of Example 2, and were composed into four replicate stacks, also including an uncoated negative control membrane. Antibodies to assay targets ErbB2, ER (estrogen receptor) and CK7 (cytokeratins 7) were respectively conjugated with CY5 fluorescent-tagged sense ODN, in the same manner as described above and shown in complex 40 on FIG. 2B. These conjugates were used to bind conjugates to tissue sections by following a conventional IHC protocol. Either one or three conjugated Abs were used per slide. After the last washing step, coated membranes were equilibrated in release/hybridization buffer (4×SSC/0.1% SDS, 50 mM beta-mercaptoethanol) and applied on the slide for a 30 min incubation at 47° C. The stacks were dissociated, membranes washed in 2×SSC/0.1% SDS, rinsed in distilled water and dried before being scanned on bed flat scanner (Typhoon) with the results shown (FIG. 17A). The data provide a comparison of the three conditions: a) The amount of conjugate bound on the slide before transfer of transfer strands (first image column); b) the amount of transfer strands transferred to capture membrane when one conjugate is applied to the tissue (rows 1-3 in image columns 2-5); and the amounts of transfer strands transferred to tissue when all three conjugates are incubated on the tissue together (row 4 in image columns 2-5). The control membrane without any antisense conjugated oligonucleotide had no signal (image column 2). Selected image areas were quantitatively measured for CY5 fluorescence using ImageQuant software and the data were plotted (FIG. 17B). In this experiment, single-staining data were proportional to multiplex data and were more intense.

### Example 9

### Further Comparison of Double Staining Triple Staining

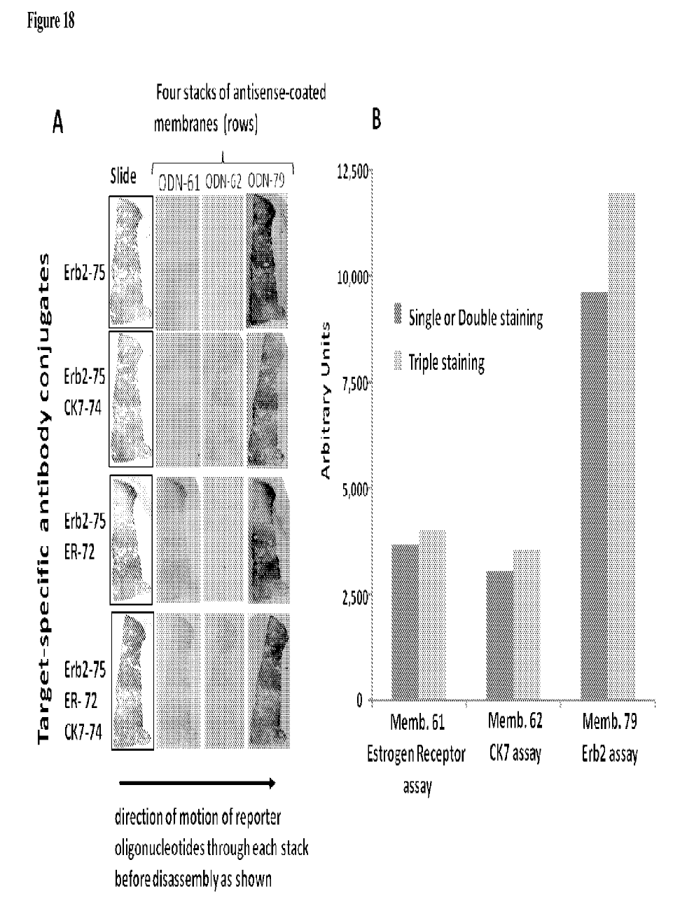

The same materials and methods of Example 8 were used, except that either one, two or three conjugates were applied together to one tissue section. The data (FIG. 18) indicate that the capture efficiency of the released ODN-cy5 to complementary membranes is not much affected by the presence of multiple conjugated antibodies. Quantitative analysis (FIG. 18B) indicated that the intensity of triple transfers (row 4) was nearly the same as that of double transfers (rows 2 and 3) or single transfers (row 1).

### Example 10

### Target Localization in Tissue Using Triple Multiplex Mode

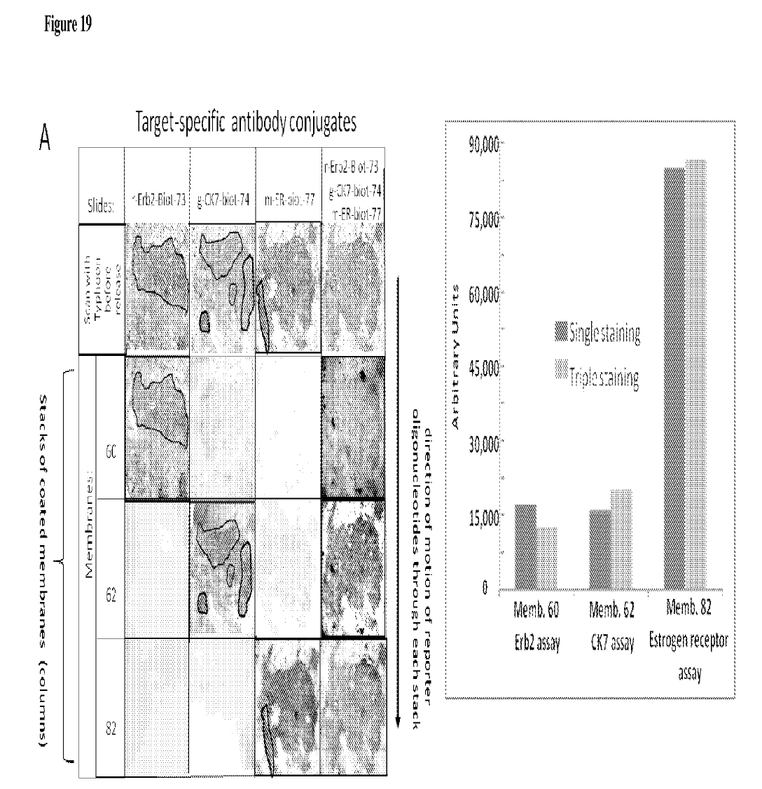

The same materials and methods of Example 8 were used. In FIG. 19A, some of the target proteins seem to have been over-expressed in those manually circumscribed areas are imaged both on the slide before transfer (row 1) and on the membranes after transfer, such as: ErbB2 on membrane 60 (row 2) and CK7 on membrane 62 (row 3). Quantitative analysis in FIG. 19B indicates that overall intensities as measured in uniplex and triplex modes were indeed comparable, although those local intensity variations were less obvious.

What is claimed is:

1. A method of analyzing biomolecules in a tissue section comprising the steps of:
providing a stack of membranes, wherein each of said membranes has a different oligonucleotide attached thereto;
providing a set of antibodies that have specific affinity to the biomolecules in the tissue section, wherein each of said antibodies has conjugated thereto a cleavable oligonucleotide that can hybridize to the oligonucleotide attached to said membranes;
applying said antibodies to said tissue section in a manner that permits the antibodies to bind to the biomolecules;
cleaving said oligonucleotides from said antibodies;
transferring said cleaved oligonucleotides from said antibodies to said membrane stack whereupon the transferred oligonucleotide binds to complimentary oligonucleotides attached to the membranes;
detecting the hybridization of the transferred oligonucleotides.

2. An oligonucleotide coated track etched membrane comprising:
a track etched membrane; and
aminated oligonucleotides covalently bound at their amino group to a carboxyl or carbonic group of the track etched membrane.

3. An oligonucleotide coated track etched membrane comprising either of the two structures: 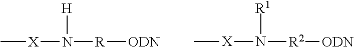
Wherein X is a structural component of the track etched membrane; N is an alkyl amine connecting the oligonucleotide to the structural component of the track etched membrane; R¹ is a substituent of the alkyl amine; and R² is a linker that may be connected to a phosphate group at a 5′ or 3′ terminal end of the oligonucleotide/oligodeoxynucleotide; and ODN is an oligodeoxynucleotide.

4. A method of capturing oligonucleotides from a sample comprising the steps of:
providing a stack of two or more track-etched membranes, wherein each of said track-etched membrane has a different oligonucleotide attached thereto that can hybridize with an oligonucleotide in the sample;
transferring said sample oligonucleotides to said track-etched membrane stack wherein the sample oligonucleotides hybridize to the oligonucleotides attached to membranes.

5. The method according to claim 4 wherein said oligonucleotide is attached to said membrane by a covalent bond.

6. The method according to claim 4, wherein said track etched membranes are selected from the group consisting of polyimide track etched membranes, polycarbonate track etched membranes, and polyethylene terephthalate track etched membranes.

7. A method of making oligonucleotide coated track etched membranes, said method comprising the steps of:
obtaining track etched membranes;
obtaining aminated oligonucleotides;
obtaining a condensing agent;
reacting said track etched membranes in the presence of said condensing agent,
thereby forming said oligonucleotide coated track etched membranes.

8. The method according to claim 7, wherein said condensing agent is a carbodiimide.

9. The method according to claim 8, wherein carbodiimide is selected from the group consisting of Dimethylaminopropyl)-N′-ethylcarbodiimide hydrochloride, 1-cyclohexyl-3-(2-morpholinoethyl)carbodiimide, and 1-benzyl-3-dimethylaminopropylcarbodiimide.

10. The method according to claim 7, wherein said track etched membranes are selected from the group consisting of polyimide track etched membranes, polycarbonate track etched membranes, and polyethylene terephthalate track etched membranes.

11. The method according to claim 8, further comprising washing said oligonucleotide bound track etched membrane in a solvent having the capability to remove virtually any adsorbed aminated oligonucleotides.

12. The method according to claim 11, wherein said solvent is a polar aprotic solvent.

13. The method according to claim 12, wherein said polar aprotic solvent comprises acetonitrile.

14. The method according claim 7, further comprising the step of degassing said track etched membranes prior to exposing them to said aminated oligonucleotides.

15. The method according to claim 14, wherein said degassing occurs under vacuum pressure.
